(19) United States
(12) Patent Application Publication
(10) Pub. No.: US 2009/0171303 A1
(43) Pub. Date: Jul. 2, 2009

(54) Fixed- or Variable-Length, Wire-Reinforced Catheter and Method of Adaptation

(76) Inventors: David A. Loiterman, Oak Brook, IL (US); Michael G. Loiterman, Yorkville, IL (US)

Correspondence Address: VEDDER PRICE P.C., 222 N. LASALLE STREET, CHICAGO, IL, 60601, US

(21) Appl. No.: 12/343,888

(22) Filed: Dec. 24, 2008

Related U.S. Application Data
(60) Provisional application No. 61/017,095, filed on Dec. 27, 2007.

(51) Int. Cl. A61L 29/16; A61M 25/00

(52) U.S. Cl. 604/265; 604/526

(57) ABSTRACT

This disclosure relates to a fixed- or variable-length, wire-reinforced catheter for use in a human body or connected to a subcutaneous port implanted in a human body for use under a cyclical internal load and method of adaptation thereof, and more particularly, to a wire-reinforced catheter of increased internal diameter or reduced external diameter of fixed or variable lengths for the optimized transportation of blood under cyclical internal load and without damage.

### CROSS-REFERENCE TO RELATED APPLICATION

The present patent application claims priority from and the benefit of U.S. Provisional Patent Application No. 61/017,095, filed Dec. 27, 2007, and entitled Wire-Reinforced Silicone Catheter, which prior application is hereby incorporated herein by reference.

### FIELD OF THE DISCLOSURE

This disclosure relates to a fixed- or variable-length, wire-reinforced catheter for use in a human body or connected to a subcutaneous port implanted in a human body for use under a cyclical internal load and method of adaptation thereof, and more particularly, to a wire-reinforced catheter of increased internal diameter or reduced external diameter of fixed or variable lengths for the optimized transportation of blood under cyclical internal load and without damage.

### BACKGROUND

Catheters are used during medical interventions in a wide variety of biomechanical applications. Small, hollow tubes are introduced within a patient's body to remove bodily fluids, circulate these fluids through external equipment, provide access for equipment, etc. For example, some catheters can be used during the dialysis process where an external device filters chemicals and compounds such as urea from blood or adjusts the volume of said chemicals and compounds in a patient.

The human heart is a cyclical pump that circulates blood through the human body by pumping at a fixed cadence. This biological pump sends cyclical pressure loads into the human body, which is capable of withstanding these pressure variations. Veins and arteries are also evolved not to swell and ultimately rupture when blood pressure is positive or collapse inwardly when blood pressure is negative. Catheters inserted in the body are often hooked up to cyclical pumps that simulate the human heartbeat to limit disruptions to the host. But such devices create a greater cyclical pressure gradient.

During renal failure, the kidneys are replaced with an external dialyzer that filters waste from the blood. Catheters are inserted into the body and connected to these external filtration machines. However, the dimensions of traditional catheters do not offer optimal laminar flow of the blood within the catheter at both the initial lower viscosity of a pre-dialysis treated blood and the subsequent higher viscosity of a post-dialysis treated blood. What is needed is a catheter where the length and diameter of the catheter are optimized to facilitate optimal laminar flow of blood at the lowest possible pressures and the highest possible flow rates at both an initial lower viscosity of a pre-dialysis treated blood and a subsequent higher viscosity of post-dialysis treated blood.

Catheters are de facto smaller in diameter than the vein or artery in which they are inserted. For the full volume of blood to be pumped, the blood must travel faster through the reduced area of the catheter; and as a consequence, greater pressure is needed to accelerate the blood through the smaller diameter. The thickness of the wall of the catheter must be minimized to reduce the acceleration of blood in the catheter, but thin-walled catheters often lack rigidity and may collapse or rupture under the pressure gradients created by the artificial pumping device. For this reason, thin-walled catheters are useful as long as their mechanical properties are not compromised by the reduction of wall thickness. What is needed is a thin-walled catheter where the wall is reinforced to withstand pressure variations even at a reduced thickness.

For example, a catheter may be inserted in the urethra when the conduit is damaged, in the abdomen in the case of an abdominal abscess, for the administration of intravenous fluids during angioplasty, angiography, or balloon septostomy, for administration of anesthetic medication, and for the subcutaneous administration of insulin or other medication for medical treatment such as chemotherapy.

FIGS. 1, 2, and 3 are taken from U.S. Pat. No. 5,041,098, which is incorporated herein by reference and is a prior art device co-invented by the inventor of the present disclosure. FIG. 1 shows the implantation in a human body of two access systems, each with a port and associated catheters. This system can be implanted within the body, i.e., in the vasculature of the chest, such that both access systems are located just beneath the epidermis and above the musculature. Further, the catheters have access to the vasculature through major vessels such as the subclavian vein down to, for example, the junction of the superior vena cava and the right atrium of the heart. FIGS. 2 and 3 show the access system with an attached catheter from the prior art via a port.

One major problem with catheter insertion is the effect of routine movement of the human body through space. Arteries and veins flex and expand to accommodate the biological functions of the body. As such, catheters must be flexible, soft, and capable of flexion and rotation while not collapsing when internal pressure is placed on the catheter (for example, during exercise) or when external pressure is placed on the catheter (for example, during sleep). Pinching may occur where opposite walls of a tube join when a catheter lacks flexibility.

In contrast, under other conditions of use, catheters must withstand pressure increases or decreases associated with pumping fluid through the tube. Dialysis patients are greatly encumbered by having to wait for extended periods of time while urine is slowly filtered from the blood. For this reason, machines must pump blood faster, thus creating significant pressure differentials. Paradoxically, flexible catheters under pressure may cave in, clog, or close, all of which can damage blood cells. What is needed is a flexible yet strong catheter capable of bending but not pinching or breaking when moved while also being capable of withstanding pressure conditions imposed upon the catheter by external equipment.

Finally, blood is a fragile liquid formed by an agglomeration of biological cells such as red and white blood cells held in suspension in a serum. Cells are small, fragile bodies in a biological serum and can be damaged if put under strain. When blood is transported in human veins, flow remains laminar and the blood pressure stays well within acceptable limits. When blood flow and velocity is increased by an external pumping mechanism, cells can be damaged if blood enters nonlaminar velocity even locally at the tip of a catheter. What is needed is a catheter capable of preventing damage to blood cells during use.

### SUMMARY

This disclosure relates to a fixed- or variable-length, wire-reinforced catheter for use in a human body or connected to a subcutaneous port implanted in a human body for use under a cyclical internal load and method of adaptation thereof, and more particularly, to a wire-reinforced catheter of increased internal diameter or reduced external diameter of fixed or variable lengths for the optimized transportation of blood under cyclical internal load and without damage.

### BRIEF DESCRIPTION OF THE DRAWINGS

Certain embodiments are shown in the drawings. However, it is understood that the present disclosure is not limited to the arrangements and instrumentality shown in the attached drawings.

FIG. 1 taken from the prior art illustrates the use of a catheter as an access system for extracorporeal blood treatment.

FIG. 2 is a top plan view of an access port known in the art with a port and a catheter attached thereto that may be used as depicted in FIG. 1.

FIG. 3 is a sectional view of the device shown in FIG. 2 as is known in the art.

FIG. 4 is an illustration of a wire-reinforced catheter according to an embodiment of the present invention connected to an access system equipped with a port.

FIG. 5 is an illustration of the wire-reinforced catheter according to an embodiment of the present invention shown in FIG. 4 with the catheter disconnected from the access system and its port.

FIG. 6 is a side sectional view of a hybrid, wire-reinforced catheter according to an embodiment of the present disclosure.

FIG. 7A is a side sectional view of a hybrid, wire-reinforced tube of the hybrid, wire-reinforced silicone catheter shown in FIG. 6 according to an embodiment of the present disclosure.

FIG. 7B is an end view of the hybrid, wire-reinforced tube of FIG. 7A according to an embodiment of the present disclosure.

FIG. 8A is a side sectional view of a hybrid cap of the hybrid, wire-reinforced catheter shown in FIG. 6 according to an embodiment of the present disclosure.

FIG. 8B is an end view of the hybrid cap of FIG. 8A according to an embodiment of the present disclosure.

FIG. 9 is a side view of a fixed-length, wire-reinforced catheter according to another embodiment of the present disclosure.

FIG. 10 is a side sectional view of a fixed-length, wire-reinforced tube of the fixed-length, wire-reinforced catheter shown in FIG. 6 according to another embodiment of the present disclosure.

FIG. 11 is detail view of an end tip of the fixed-length, wire-reinforced tube as shown in FIG. 10 according to another embodiment of the present disclosure.

FIG. 12 is an end view of the fixed-length, wire-reinforced tube of FIG. 10 according to another embodiment of the present disclosure.

FIG. 13 is a side view of the handle of the fixed-length, wire-reinforced tube of FIG. 10 according to another embodiment of the present disclosure.

FIG. 14 is a side elevation view of the handle of FIG. 13 according to another embodiment of the present disclosure.

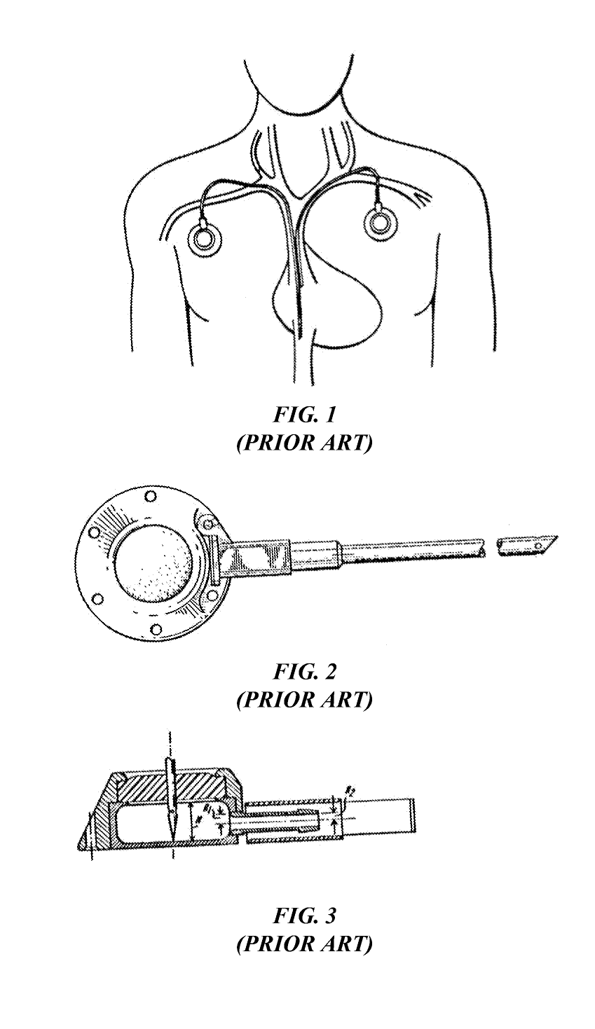

### DETAILED DESCRIPTION OF THE INVENTION

For the purposes of promoting and understanding the principles disclosed herein, reference is now made to the preferred embodiments illustrated in the drawings, and specific language is used to describe the same. It is nevertheless be understood that no limitation of the scope of the invention is hereby intended. Such alterations and further modifications in the illustrated devices and such further applications of the principles disclosed and illustrated herein are contemplated as would normally occur to one skilled in the art to which this disclosure relates.

One of ordinary skill in the art recognizes the plurality and diversity of applications of the wire-reinforced catheter described herein, along with the variety of minute adjustments that must be made to attach the catheter to different ports or external devices.

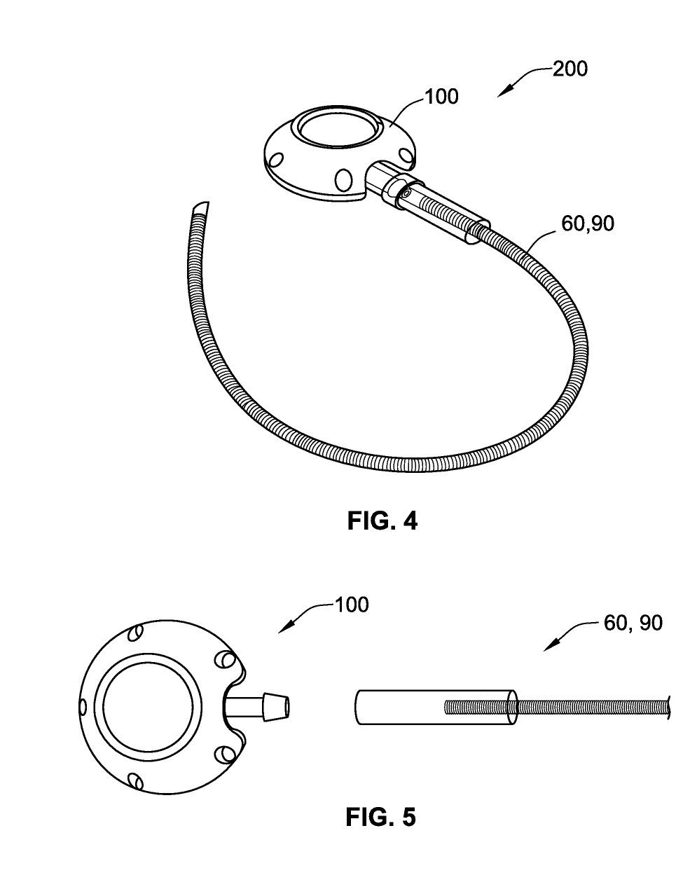

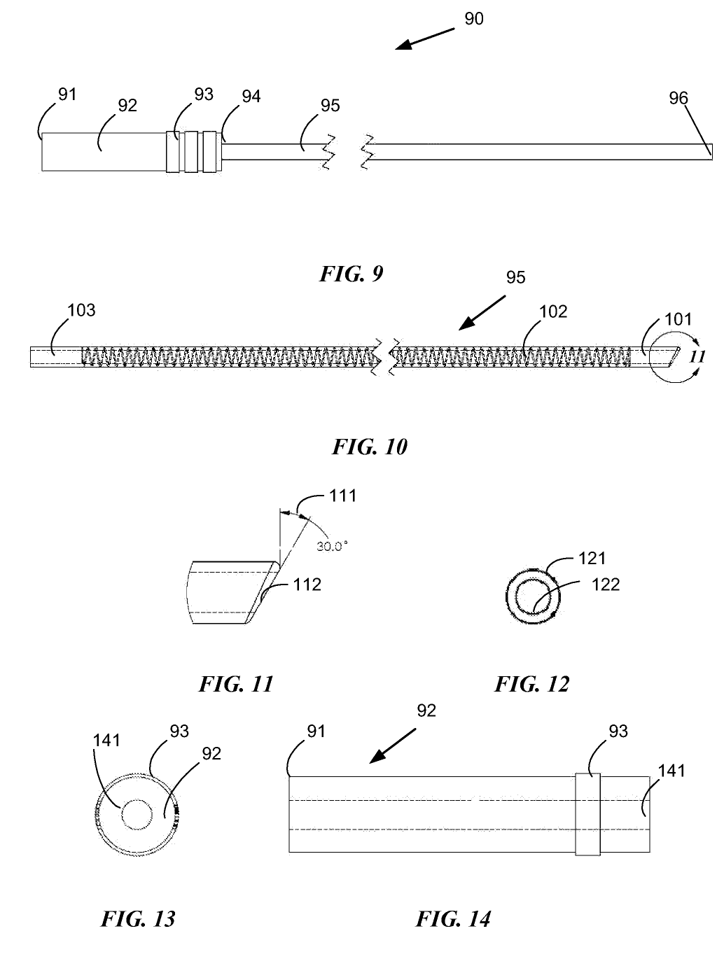

FIG. 4 is an illustration of a wire-reinforced catheter according to an embodiment of the present invention connected to an access system equipped with a port. FIG. 5 is an illustration of the wire-reinforced catheter according to an embodiment of the present invention shown in FIG. 4 with the catheter disconnected from the access system and its port. FIGS. 6 to 8B show a first embodiment of the present disclosure where the length of the tube is not fixed and can be cut to length as needed according to a method of cutting a variable-length catheter. FIGS. 9 to 14 show another embodiment of the present disclosure where the length of the tube is fixed. Patients vary in overall size and must be accommodated by catheters of different lengths. The catheter distal ends 71 and 96 as shown in FIGS. 6 and 9, respectively, must be made to rest at a precise location within a patient's body depending on the intended use of the catheter. In a preferred embodiment, catheters of approximately 17.12 cm, 19.62 cm, and 22.23 cm in overall length are contemplated that correspond to three optimal lengths based on the mean measured value (e.g., 20 cm for one contemplated use) with standard deviations of 1.5 cm, and a standard deviation of 6 cm to accommodate over 99% of patients. In this embodiment, catheters of 14 to 26 cm and internal diameters of 1 to 6 mm are contemplated, and the three preferred lengths above allow for the accommodation of each patient within a narrow margin of error. While a preferred embodiment is shown, what is contemplated is a range of lengths and diameters to accommodate the different functions described above.

To improve catheters overall, the pressure loss associated with the flow of fluid within the catheter must be reduced. This can be achieved by increasing the internal diameter of the catheter, by shortening the length of the catheter, by smoothing the flow interfaces and edges, etc. Shorter catheters with narrower tube walls are improvements over the prior art. What is contemplated is the thinning of the tube wall made of silicone by molding within a portion of the tube a reinforcement in the shape of an endless spiral wire made of biologically inert material. In another embodiment, polyurethane is used instead of silicone. The reinforcement is located at the portion of the tube where the inner pressure caused by the external device is maximum and where the catheter must be bent or curled to a specific position for ease of access. In one preferred embodiment, the catheter curl is approximately 5 cm or about one-quarter of the overall length of the catheter.

A spring is a suitable form of reinforcement to mold within a tube. Other reinforcement elements, such as a ribbon, a mesh, or any other structural material capable of taking forces, are also contemplated. A spring does not decrease significantly the resistance of the tube in which it is placed. A series of adjacent rings, also called a pitch, can move apart and bend the opposite side of the spring without effort. Springs made of wire offer resistance when they are placed in traction, as is the case when the tube is pressurized and the silicone matrix expands. Under pressure, the strain normally felt by the silicone or other material is transferred onto the spiral wire once the expansion has reached a value sufficient to place the spiral wire under strain. As a consequence, the silicone matrix, the polyurethane matrix, or the matrix formed of any other biocompatible material is protected and can be reduced in thickness to withstand pressure.

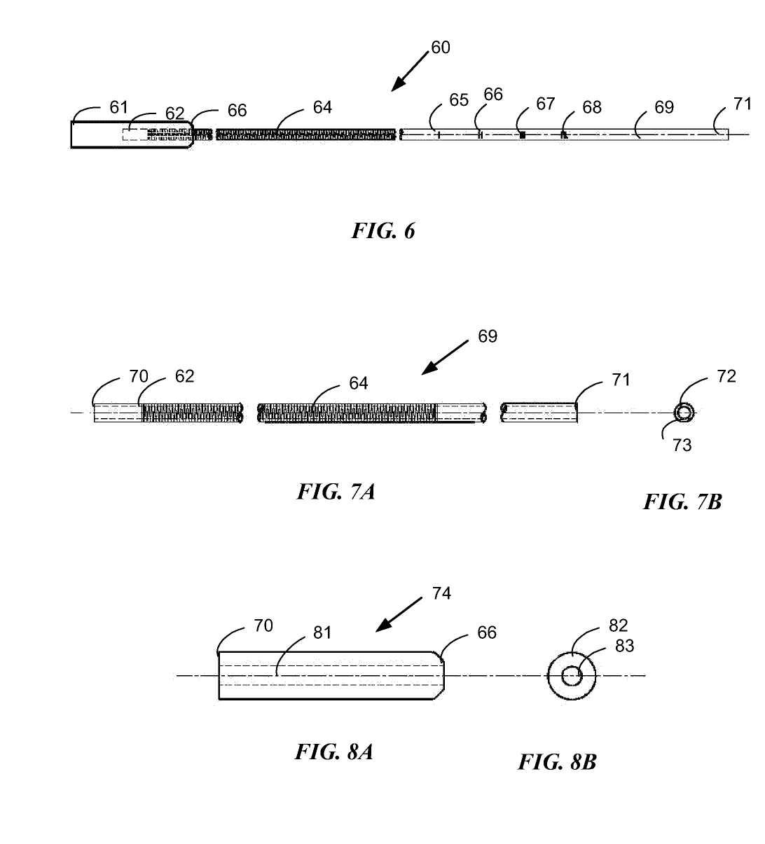

FIG. 6 is a side sectional view of a hybrid, wire-reinforced catheter 60 according to an embodiment of the present disclosure. The catheter 60 includes a silicone tube 69 with an inside diameter 73 and an outside diameter 72 as shown in FIG. 7B. The tube 69 includes a proximate end 70 and a distal end 71 in opposition thereto. Flow of a liquid from the proximate end 70 and the distal end 71 occurs within the opening made by the inside diameter 73. In one preferred embodiment, the hybrid tube 69 is 50.8 cm in length with an internal diameter of 0.063 to 0.068 inch and an external diameter of 0.1 inch. The radius of the inside diameter 73 is calibrated along with the length of the tube 69 to offer optimal laminar flow of the blood within the tube at both the initial lower viscosity of a pre-dialysis treated blood and the higher viscosity of a post-dialysis treated blood.

While silicone is described as a preferred biocompatible material matrix for the construction of the catheter tube, any, flexible and biologically inert medium is contemplated, such as, silicone or polyurethane. While one range of internal and external diameter of the tube 69 is described as the preferred embodiment, what is contemplated is the use of any external diameter capable of insertion within the vein or passageway of a body, and an internal diameter capable of maintaining an acceptable flow within the tube 69 without significantly hindering the fluid. For example, when a liquid solution is transported in the tube 69, the internal diameter can be very small, but when blood or other fluids with large cells or compounds are transported, the internal diameter must be sufficiently large to maintain unobstructed and nondamaging flow of the elements. For dialysis treatment, blood is transported in the tube 69. Other fluids may also be transported based on the application of the catheter 60.

A cap 74 is also shown as a cylindrical body made of silicone in one embodiment or any other biocompatible material such as, for example, polyurethane, that includes a port connection end 70 and a tube connection end 66 in opposition thereto. The cap 74 further includes an inner opening 81 as shown in FIG. 8A, which is bonded at the tube 69 at the connection end 66 to the outside diameter 72. Bonding is conducted between pieces by known bonding techniques to prevent the formation of air pockets, voids, and leaks. As shown in FIG. 6, a significant portion of the cap 74 is slid over a tube connection end 66 to ensure proper bonding. A first portion of the tube 69 located between the proximate end 70 and the distal end 71 includes a spiral wire 64 within the silicone of the tube 69 between the inside diameter 73 and the outside diameter 72.

FIG. 7A shows which portion of the tube 69 may be reinforced with the spiral wire 64. In the first embodiment, the entire portion where the tube 69 leaves the cap 74 to a distant portion of the tube 69 is reinforced. Further, FIG. 6 shows how the hybrid, wire-reinforced catheter 60 can be sized at different lengths either by printing marks or by creating bumps on the outside surface of the silicone when molded. In another embodiment, a guidewire (not shown) is inserted in a patient and has reverse position from the surface markings of the tube 69 calibrated to the different desired lengths to help determine the needed length of catheter 60. Once the needed length is determined, a cutting device is used to remove the end portion of the tube 69. What is also contemplated is the creation of an end tip with an angle or with smoothed edges done by different technical processes, such as glazing. If ink is used for markings, the ink must be implantable ink and all grades of silicone must be certified for biomedical use. As a result of use of the guidewire (not shown) within the body, the markings made on the surface of the guidewire (not shown) are in reverse order from the markings made on the tube 69 because insertion is measured after insertion of the guidewire (not shown).

FIG. 9 is a side view of a fixed-length, wire-reinforced catheter 90 according to another embodiment of the present disclosure. FIG. 9 shows how a tube 95 having a proximate end 94 and a distal end 96 in opposition can be bonded to a cap 92. The distal end 96 includes a tip 101 as shown in FIG. 10 without wire 102 that is cut at an angle as shown in the detail of FIG. 11. In a preferred embodiment, the tip is cut at an angle 30° from vertical and the edges at both the internal diameter 122 and the external diameter 121 are made smooth by glazing. Instead of using a marking on the tube 95, the cap 92 may include rings 93 or any other indication to indicate the type of fixed-length, wire-reinforced catheter 90 in use. The use of rings 93 coded to correspond to the different available lengths of the tube 69 is contemplated and can be manually confirmed by the user. While one type of marking is described, what is contemplated is the use of any marking capable of recognition by a physician or other medical practitioner using the catheter either through visual or tactile recognition, including colors and shapes. The cap 92 also includes an inside opening 141 for the passage of the fluid moving through the tube 95. Finally, the cap 92 or 74 is of an internal diameter such that it can be slid over a port (not shown) having a lock-in feature such that an external lip with an edge on the outer rim of the port facilitates the entry of the cap and prevents accidental removal of the cap from the port.

In yet another embodiment, a coating such as heparin or other drugs is placed upon the external surface of the tube 69 and/or the inside surface or tip 71 of the tube 69 to prevent infection or clotting. Superficial coating is preserved and protected by a series of measures, including the use of sterile environments, transport bags, and glove manipulation.

What is also contemplated is a method for fixing the length of a variable-length, wire-reinforced catheter as described above where first a guidewire is inserted from the proximate end into the patient until the desired destination is reached by the distal end, then a note is made associated with the closest marking entered into the patient, then the guidewire is removed from the patient before the tube is cut at the distal end of the tube, at the discretion of the physician, such that the distance matches the closest marking entered into the patient. For more precise placement, the port and catheter assembly can be moved back and forth by some degree of latitude by the physician to achieve optimal placement of catheter tip.

In another embodiment, what is also contemplated is a method for selecting a fixed-length, wire-reinforced silicone catheter from a plurality of fixed-length, wire-reinforced catheters, each having a different length, introducing in a patient a guidewire with a series of markings located along the length of the tool to determine the needed length of the catheter to be inserted, and selecting from a plurality of fixed-length, wire-reinforced catheters, each with a different marking associated with the markings of the guidewire, the catheter with the number of rings on the cap associated with the last series of rings of the guidewire entered into the patient, i.e., the one with the greatest but not superior to the needed length.

It is understood that the preceding is merely a detailed description of some examples and embodiments of the present invention and that numerous changes to the disclosed embodiments can be made in accordance with the disclosure made herein without departing from the spirit or scope of the invention. The preceding description, therefore, is not meant to limit the scope of the invention but to provide sufficient disclosure to one of ordinary skill in the art to practice the invention without undue burden.

What is claimed is:

1. A wire-reinforced catheter, comprising:
a tube with an inside diameter and an outside diameter having a thickness, the tube having a proximate end and a distal end in opposition thereto; and
a cap with a port connection end and a tube connection end, the cap further including an inner opening bonded at the tube connection end to the outside diameter of the tube at the proximate end,
wherein a first portion of the tube located between the proximate end and the distal end includes in the thickness a spiral wire reinforcement.

2. The wire-reinforced catheter of claim 1, wherein the tube is coated by an anticoagulant.

3. The wire-reinforced catheter of claim 2, wherein the anticoagulant is heparin.

4. The wire-reinforced catheter of claim 1, wherein the tube is coated by a substance to prevent infection.

5. The wire-reinforced catheter of claim 1, wherein the distal end includes an angled tip.

6. The wire-reinforced catheter of claim 1, wherein the distal end includes a tip with smoothed edges obtained by glazing.

7. The wire-reinforced catheter of claim 1, wherein the tube is made of a biocompatible matrix.

8. The wire-reinforced catheter of claim 7, wherein the biocompatible matrix is a silicone-based matrix.

9. The wire-reinforced catheter of claim 8, wherein the biocompatible matrix is a polyurethane.

10. The wire-reinforced catheter of claim 1, wherein the bonded portion of the inner opening to the outside diameter of the tube at the proximate end is a significant portion of the length of the inner opening.

11. A reinforced catheter for the transportation of blood, comprising:
a tube with an inside diameter and an outside diameter having a thickness, the tube having a proximate end and a distal end in opposition thereto,
a liquid in contact with the inside diameter of the tube; and
a cap with a port connection end and a tube connection end, the cap further including an inner opening bonded at the tube connection end to the outside diameter of the tube at the proximate end,
wherein a first portion of the tube located between the proximate end and the distal end includes in the thickness a reinforcement element, and wherein the fluid is blood.

12. The reinforced catheter for the transportation of blood of claim 11, wherein the reinforcement element is selected from a group consisting of a spring, a ribbon, and a mesh.

13. A variable-length, wire-reinforced silicone catheter, comprising:
a silicone tube with an inside diameter and an outside diameter having a thickness, the silicone tube having a proximate end and a distal end in opposition thereto distant by a length; and
a cap with a port connection end and a tube connection end, the cap further including an inner opening bonded at the tube connection end to the outside diameter,
wherein a first portion of the tube located between the proximate end and the distal end includes a spiral wire within the thickness in a portion of the total length, and wherein the length is divided in several successive marked sections.

14. The variable-length, wire-reinforced silicone catheter of claim 13, wherein the markings are printed on the outside diameter of the silicone tube in implantable ink.

15. The variable-length, wire-reinforced silicone catheter of claim 13, wherein the markings are molded bumps on the outside diameter of the silicone tube.

16. The variable-length, wire-reinforced silicone catheter of claim 13, where the spiral wire is within the thickness of the portion of the total length between the proximate end and a first of the several successive marked sections.

17. The variable-length, wire-reinforced silicone catheter of claim 13, wherein the successive markings are separated by marks located at distances of approximately 17.12 cm, approximately 19.62 cm, and approximately 22.23 cm from the proximate end.

18. The variable-length, wire-reinforced silicone catheter of claim 17, wherein the tube has an internal diameter of 1 to 6 mm.

19. The variable-length, wire-reinforced silicon catheter of claim 17, wherein the tube has a length of 50.8 cm and an internal diameter of 0.063 to 0.068 inch.

20. The variable-length, wire-reinforced catheter of claim 13, wherein the outside diameter is coated by an anticoagulant.

21. The variable-length, wire-reinforced catheter of claim 13, wherein the anticoagulant is heparin.

22. The variable-length, wire-reinforced catheter of claim 13, wherein the distal end includes an angled tip.

23. The variable-length, wire-reinforced catheter of claim 13, wherein the distal end includes a tip with smoothed edges obtained by glazing.

24. The variable-length, wire-reinforced catheter of claim 13, wherein the bonded portion of the inner opening to the outside diameter of the tube at the proximate end is a significant portion of the length of the inner opening.

25. A fixed-length, wire-reinforced silicone catheter, comprising:
a silicone tube with an inside diameter and an outside diameter having a thickness, the silicone tube having a proximate end and a distal end in opposition thereto distant by a length; and
a cap with a port connection end and a tube connection end, the cap further including an inner opening bonded at the tube connection end to the outside diameter and at least a marking,
wherein a first portion of the tube located between the proximate end and the distal end includes a spiral wire within the thickness in a portion of the total length, and wherein the marking is associated with a specific length.

26. The fixed-length, wire-reinforced catheter of claim 25, wherein the tube is coated by an anticoagulant.

27. The fixed-length, wire-reinforced catheter of claim 26, wherein the anticoagulant is heparin.

28. The fixed-length, wire-reinforced catheter of claim 25, wherein the distal end includes an angled tip.

29. The fixed-length, wire-reinforced catheter of claim 25, wherein the distal end includes a tip with smoothed edges obtained by glazing.

30. The fixed-length, wire-reinforced catheter of claim 25, wherein the bonded portion of the inner opening to the outside diameter of the tube at the proximate end is a significant portion of the length of the inner opening.

31. The fixed-length, wire-reinforced catheter of claim 25, wherein the marking is at least a ring.

32. The fixed-length, wire-reinforced catheter of claim 25, wherein a configuration of one, two, and three concentric rings is associated with a silicone tube length of approximately 17.12, approximately 19.62, and approximately 22.23 cm, respectively.

33. A method of fixing the length of a variable-length, wire-reinforced catheter made of a tube with an inside diameter and an outside diameter having a thickness, the tube having a proximate end and a distal end in opposition thereto distant by a length; and a cap with a port connection end and a tube connection end, the cap further including an inner opening, wherein a first portion of the tube located between the proximate end and the distal end includes a reinforcement element within the thickness in a portion of the total length, and wherein the length is divided in several successive marked sections, the method comprising the steps of:
inserting a tube from the proximate end into the patient until the desired destination is reached by the distal end;
noting the closest marking entered into the patient;
removing the tube from the patient;
cutting the distal end of the tube based on the note made; and
attaching the cap to a port.

34. A method of selecting a fixed-length, wire-reinforced catheter from a plurality of fixed-length, wire-reinforced catheters, each having a different length, each catheter made of a tube with an inside diameter and an outside diameter having a thickness, the tube having a proximate end and a distal end in opposition thereto distant by a length; and a cap with a port connection end and a tube connection end, the cap further including an inner opening bonded at the tube connection end to the outside diameter and at least a marking, wherein a first portion of the tube located between the proximate end and the distal end includes a spiral wire within the thickness in a portion of the total length, and wherein the marking is associated with a specific length, the method comprising the steps of:
introducing in a patient a guidewire with a series of markings located along the length of the wire to determine the needed length of the catheter to be inserted; and
selecting from a plurality of fixed-length, wire-reinforced catheters each with a different marking associated with the markings of the guidewire the catheter with the number of rings on the cap associated with the last series of rings of the guidewire entered into the patient, the one with the greatest but not superior to the needed length.
